SEQUENCE LISTING (23 sequences)

SEQ ID NO: 1 - DNA, 9 bp, Artificial Sequence
  Description of Artificial Sequence Synthetic oligonucleotide
gaagaagaa

SEQ ID NO: 2 - DNA, 12 bp, Artificial Sequence
  Description of Artificial Sequence Synthetic oligonucleotide
gaagaagaagaa

SEQ ID NO: 3 - DNA, 15 bp, Artificial Sequence
  Description of Artificial Sequence Synthetic oligonucleotide
gaagaagaagaagaa

SEQ ID NO: 4 - DNA, 18 bp, Artificial Sequence
  Description of Artificial Sequence Synthetic oligonucleotide
gaagaagaagaagaagaa

SEQ ID NO: 5 - DNA, 99 bp, Artificial Sequence
  Description of Artificial Sequence Synthetic oligonucleotide
gaagaagaagaagaagaagaagaagaagaagaagaagaagaagaagaagaagaagaagaagaagaagaagaagaagaagaagaagaagaagaagaagaa

SEQ ID NO: 6 - DNA, 528 bp, Artificial Sequence
  Description of Artificial Sequence Synthetic oligonucleotide
gaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaa

SEQ ID NO: 7 - DNA, 6 bp, Artificial Sequence
  Description of Artificial Sequence Synthetic oligonucleotide
aagaag

SEQ ID NO: 8 - DNA, 9 bp, Artificial Sequence
  Description of Artificial Sequence Synthetic oligonucleotide
aagaagaag

SEQ ID NO: 9 - DNA, 15 bp, Artificial Sequence
  Description of Artificial Sequence Synthetic oligonucleotide
aagaagaagaagaag

SEQ ID NO: 10 - DNA, 18 bp, Artificial Sequence
  Description of Artificial Sequence Synthetic oligonucleotide
aagaagaagaagaagaag

SEQ ID NO: 11 - DNA, 19 bp, Artificial Sequence
  Description of Artificial Sequence Synthetic oligonucleotide
aggaggaggtggaggagga

SEQ ID NO: 12 - DNA, 61 bp, Artificial Sequence
  Description of Artificial Sequence Synthetic oligonucleotide
gccttacggttacacttgatgaagaagaagaagaagaattcgcaatgccattgcgctatga

SEQ ID NO: 13 - DNA, 21 bp, Artificial Sequence
  Description of Artificial Sequence Synthetic oligonucleotide
gtacctactagtccagtgtgg

SEQ ID NO: 14 - DNA, 21 bp, Artificial Sequence
  Description of Artificial Sequence Synthetic oligonucleotide
ctcgatatctgcagaattgcc

SEQ ID NO: 15 - DNA, 18 bp, Artificial Sequence
  Description of Artificial Sequence Synthetic oligonucleotide
ggccaacatggtgaaacc

SEQ ID NO: 16 - DNA, 20 bp, Artificial Sequence
  Description of Artificial Sequence Synthetic oligonucleotide
gtagctgggattacaggcgc

SEQ ID NO: 17 - DNA, 20 bp, Artificial Sequence
  Description of Artificial Sequence Synthetic oligonucleotide
cttgttggaatgcagttgga

SEQ ID NO: 18 - DNA, 18 bp, Artificial Sequence
  Description of Artificial Sequence Synthetic oligonucleotide
ggtttctccggtcccaat

SEQ ID NO: 19 - DNA, 18 bp, Artificial Sequence
  Description of Artificial Sequence Synthetic oligonucleotide
ggaggaggtggaggagga

SEQ ID NO: 20 - DNA, 36 bp, Artificial Sequence
  Description of Artificial Sequence Synthetic oligonucleotide
gaatccgaatccgaatccgaatccgaatccgaatcc

SEQ ID NO: 21 - DNA, 20 bp, Artificial Sequence
  Description of Artificial Sequence Synthetic oligonucleotide
cagaggaaacgctggactct

SEQ ID NO: 22 - DNA, 20 bp, Artificial Sequence
  Description of Artificial Sequence Synthetic oligonucleotide
agccagatttgcttgtttgg

SEQ ID NO: 23 - DNA, 36 bp, Artificial Sequence
  Description of Artificial Sequence Synthetic oligonucleotide
ggatccggatccggatccggatccggatccggatcc

(19) United States
(12) Patent Application Publication
(10) Pub. No.: US 2006/0270727 A1
(43) Pub. Date: Nov. 30, 2006

(54) Small molecule therapeutics and uses therefor

(76) Inventors: Christian Melander, Raleigh, NC (US); Ryan Burnett, San Diego, CA (US); Peter B. Dervan, San Marino, CA (US); Joel M. Gottesfeld, Del Mar, CA (US)

Correspondence Address: Richard J. Warburg;Foley & Lardner LLP, P.O. Box 80278, San Diego, CA, 92138-0278, US

(21) Appl. No.: 11/416,381

(22) Filed: May 2, 2006

Related U.S. Application Data
(60) Provisional application No. 60/677,441, filed on May 3, 2005.

(51) Int. Cl. A61K 31/4178

(52) U.S. Cl. 514/397

(57) ABSTRACT

The invention provides polyamides which bind genes having expanded oligonucleotide repeat sequences, which binding modulates transcription. The invention further provides methods of modulation of the transcription of such genes, and the use of polyamides as therapeutic agents to treat diseases associated with such genes.

### CROSS REFERENCE TO RELATED PATENT APPLICATIONS

This application claims the benefit of U.S. Provisional App. No. 60/677,441, filed May 3, 2005, entitled “Small Molecule Therapeutics for Friederich's Ataxia,” which is hereby incorporated by reference in its entirety and for all purposes.

This invention was made with government support under Grant Numbers R37 GM027681 and R21 NS048989. The government has certain rights in this invention.

### FIELD OF THE INVENTION

The invention relates to the field of polyamides which bind DNA having oligonucleotide repeat sequences. The invention further relates to modulation of the transcription of such DNA, and the use of polyamides as therapeutic agents to treat diseases associated with such DNA.

### BACKGROUND OF THE INVENTION

Oligonucleotide (e.g., dinucleotide, trinucleotide, tetranucleotide, pentanucleotide, and hexanucleotide) repeat disorders (e.g., trinucleotide repeat disorders) are due to genomic stretches of DNA (i.e., deoxyribonucleic acid) that contain an oligonucleotide repeat sequence (i.e., “repeat”) which is contiguously repeated (e.g., as many as 1700 times, or even more). As appreciated by one of skill in the art, recitation of an oligonucleotide sequence herein also contemplates the (Watson-Crick) complementary sequence, which of necessity is present in the opposite sense strand of duplex DNA within a genome. The term “trinucleotide repeat” refers to a trinucleotide (e.g., GAA, and the like) that is multiply repeated in a contiguous region in a gene. Expansion and hyper-expansion can occur within both introns and exons of a gene as well as regions of the genome not associated with a gene.

Many diseases, for example diseases discussed herein, are characterized by expanded oligonucleotide repeat sequences at different locations and degrees of expansion within the genome. As well known in the art, such repeats can occur throughout all genomic sequences. However, if a repeat is present in a gene, expansion of the repeat can result in a defective gene product and associated disease. Additionally, the presence of a repeat expansion in a gene can reduce transcription of the gene, leading to disease due to lack of the associated protein gene product (i.e., loss-of-function). As an example of a loss-of-function disease associated with hyper-expansion, 98% of humans suffering from Friedreich's ataxia have a hyper-expansion of a GAA triplet (i.e., trinucleotide) in the first intron of the frataxin gene. Without wishing to be bound by theory, it is generally understood that hyper-expansion of the GAA triplet in the human FXN gene results in decreased transcription and resulting lower levels of frataxin, which decrease results in disease.

### SUMMARY OF THE INVENTION

The present invention provides compounds which bind DNA having a contiguously repeated oligonucleotide sequence (i.e., oligonucleotide repeat sequence), compositions containing these compounds, and methods of use of these compounds. The compounds contemplated by the invention are polyamides (definition below) which bind in a sequence specific manner to oligonucleotide repeat sequences which are expanded within specific genes. By virtue of binding to the oligonucleotide repeat sequence, the polyamides of the invention modulate transcription of the specific genes. The modulation can be either an increase in transcription, or a decrease in transcription. The invention additionally provides methods of modulating transcription of specific genes having expanded oligonucleotide repeat sequences. Additionally, the invention provides methods for treating a subject suffering from a disease associated with a gene having an expanded oligonucleotide repeat sequence.

In a first aspect, the invention provides a polyamide which binds a gene (i.e., the “target gene”) and thereby modulates the transcription of that gene, wherein the target gene includes a plurality of repeats (i.e., expansion or hyper-expansion) of an oligonucleotide sequence (for example 5, 6, 10, 15, 20, 25, 30, 35, 36, 66, 67, 100, 200, 500, 1000, 1500, 1700, or even more copies) at which the polyamide binds. The terms “expansion” and “repeat expansion” refer to the presence of contiguously repeated oligonucleotide sequences in a gene. The term “hyper-expansion” refers to a level of expansion greater than typically observed in a population. For example, whereas typical alleles may have an expansion of 6-34 repeats, a hyper-expanded allele may include from 66-1700 repeats, or even more.

The term “polyamide” refers to polymers of linkable units chemically bound by amide (i.e., CONH) linkages; optionally, polyamides include chemical probes conjugated therewith. The term “linkable unit” refers to methylimidazoles, methylpyrroles, and straight and branched chain aliphatic functionalities (e.g., methylene, ethylene, propylene, butylene, and the like) which optionally contain nitrogen substituents, and chemical derivatives thereof. The aliphatic functionalities of linkable units can be provided, for example, by condensation of β-alanine or dimethylaminopropylaamine during synthesis of the polyamide by methods well known in the art. The term “chemical derivatives” refers to N-alkyl (e.g., N-methyl), aralkyl, or heterocycloalkyl substitution at any atom available to produce a stable compound. Linkable units are typically supplied as amino acids, desamino acids, or descarboxy amino acids prior to amide bond formation by condensation methods well known in the art to form linking amide groups. The term “amino acid” refers to an organic molecule containing both an amino group (NH₂) and a carboxylic acid (COOH). The term “desamino” refers to an amino acid from which the amino functionality has been removed. The term “descarboxy” refers to an amino acid from which the carboxylic acid functionality has been removed.

The term “binds” refers to formation of a complex between two or more molecules to a statistically greater degree than would be expected for non-interacting molecules; complexes so formed may include covalent bonding or non-covalent bonding, for example without limitation, hydrogen bonding, between two or more of the molecules of the complex. Methods for the detection of complexes involving DNA are well known in the art. Binding is characterized by a dissociation constant, K_{D}, well known in the art.

The term “chemical probe” refers to chemical functionalities having fluorescent, spectroscopic, or radioactive properties that facilitate location and identification of polyamides functionalized (i.e., covalently bonded) by such chemical probes. An example fluorescent chemical probe is the dye BODIPY, well known in the art. Methods of conjugating chemical probes to polyamides of the invention are well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences. The terms “nucleic acid” and “nucleotide” refer to ribonucleotide and deoxyribonucleotide, and analogs thereof, well known in the art.

The term “transcription,” well known in the art, refers to the synthesis of RNA (i.e., ribonucleic acid) by DNA-directed RNA polymerase. The term “modulate transcription” and like terms refer to a change in transcriptional level which can be measured by methods well known in the art, for example methods directed at the assay of mRNA, the product of transcription. In certain embodiments, modulation is an increase in transcription. In other embodiments, modulation is a decrease in transcription.

In another aspect, the invention provides a method for modulating the transcription of a gene, which gene includes multiple copies (i.e., expansion) of a oligonucleotide repeat sequence. The modulation is effected by contacting the gene with a polyamide which binds the oligonucleotide repeat sequence, and thereby modulates the transcription of the gene. Without wishing to be bound by theory, it is well understood that increasing the availability of DNA to DNA-directed RNA polymerase can increase the level of transcription of a gene. Conversely, sequestration of DNA can result in lower levels of transcription.

In another aspect, the invention provides a method for treating a subject suffering from a disease which is associated with a gene, which gene includes an expansion of an oligonucleotide repeat sequence. The method of treatment includes administering to the subject a therapeutically effective amount of a polyamide which binds to the oligonucleotide repeat sequence forming the expansion, thereby modulating transcription of the gene. The term “disease associated with a gene” refers to a causative or putative relationship between a feature of the gene and the presence of the disease. The term “feature of a gene” refers to primary sequence features (e.g., point mutation, expansion of an oligonucleotide repeat sequence within the gene, and the like), and to the effect such primary sequence features have on higher-order structure of a gene. The term “higher-order structure of a gene” refers to formation of regions of single-stranded DNA, duplex DNA (e.g., B-DNA, non B-DNA, Z-DNA), triplex DNA, intramolecular “sticky” DNA, supercoiling, heterochromatin formation, and the like, all well known in the art. In preferred embodiments, the subject is a human.

### BRIEF DESCRIPTION OF THE FIGURE

FIG. 1: The chemical structures of polyamides FA1-FA4, BODIPY, and BODIPY-conjugated FA 1-FA4.

### DETAILED DESCRIPTION OF THE PREFERRED EMBODIMENTS

In certain embodiments, the invention provides polyamides which bind a target gene and thereby modulate the transcription of that gene, wherein the target gene includes a plurality of repeats (i.e., expansion or hyper-expansion) of an oligonucleotide repeat sequence, and wherein the polyamide binds at one or more of the oligonucleotide repeat sequences. In certain embodiments, the target gene to which polyamides of the invention bind is DRPLA, HD, AR, ATXN1, ATXN2, ATXN3, CACNA1A, ATNX7, FMR1, FMR2, FXN, DMPK, SCA8, SCA10, SCA12, or ZNF9. In certain embodiments, the target gene is of non-human origin and is understood to correspond to one of the genes recited herein. The correspondence of human and non-human genes is determined by virtue of sequence identity by methods well known in the art. The term “DRPLA” refers to the gene having human locus 12p13.31, which is associated with dentatorubral-pallidoluysian atrophy. The term “HD” refers to the gene having human locus 4p16.3, which codes for the huntingtin protein. A hyper-expansion of the trinucleotide CAG within HD is associated with Huntington Disease. The term “AR” refers to the androgen receptor gene having human locus Xq11.2-q12, which is associated with spinobulbar muscular atrophy (i.e., Kennedy disease). The term “ATNX1” refers to the gene having human locus 6p23, mutation of which is associated with spinocerebellar ataxia type 1, which mutation is a CAG expansion within the coding region of the gene. The term “ATXN2” refers to the gene having human locus 12q24.1, mutation of which is associated with spinocerebellar ataxia type 2, which mutation is a CAG expansion within the coding region of the gene. The term “ATXN3” refers to the gene having human locus 14q24.3-q31, mutation of which is associated with spinocerebellar ataxia type 3 (i.e., Machado-Joseph disease), which mutation is an expansion of a CAG repeat. The term “CACNA1A” refers to the gene having human locus 19p13, mutation of which is associated with spinocerebellar ataxia type 6, which mutation is an expansion of a CAG repeat. The term “ATXN7” refers to the gene having human locus 3p21.1-p12, mutation of which is associated with spinocerebellar ataxia type 7, which mutation is an expansion of a CAG repeat. The term “FMR1” refers to the gene having human locus Xp27.3, mutation of which is associated with Fragile X syndrome, which mutation includes an expansion of a CGG repeat. The term “FMR2” refers to the gene having human locus Xq28, mutation of which is associated with mental retardation (i.e., Fragile XE syndrome). The term “FXN” refers to the gene having human locus 9q13-q21.1, mutation of which is associated with Friedreich's ataxia. The term “DMPK” refers to the gene having human locus 19q13.2-q13.3, mutation of which is associated with myotonic dystrophy, mutation of which is an expansion of a CTG trinucleotide repeat. The term “SCA8” refers to the gene having human locus 19q21, mutation of which is associated with spinocerebellar ataxia type 8, which mutation is an expansion of a CTG trinucleotide repeat. The terms “SCA10” and “ATXN10” refer to the gene having human locus 22q13, mutation of which is associated with spinocerebellar ataxia type 10, which mutation is an expansion of the pentanucleotide ATTCT. The term “SCA12” refers to the gene having human locus 5q31-q33, mutation of which is associated with spinocerebellar ataxia type 12, which mutation is an expansion of the trinucleotide CAG. The term “ZNF9” refers to the gene having human locus 3q 13.3-q24, mutation of which is associated with myotonic dystrophy of type 2, which mutation is an expansion of the tetranucleotide CCTG in intron 1 of ZNF9. In preferred embodiments, the gene bound by polyamides of the invention is FXN.

In certain embodiments, the oligonucleotide sequence which is subject to expansion consists of 3, 4, or 5 repeated nucleotides. Examples of oligonucleotide sequences contemplated by the invention include, without limitation, CGG, GCC, GAA, CTG, CAG, CCTG, and ATTCT. In some embodiments, the oligonucleotide sequences include, without limitation, CGG, GCC, GAA, CTG, and CAG. In other embodiments, the oligonucleotide sequence includes, without limitation, CCTG. In further embodiments, the oligonucleotide sequence includes, without limitation, ATTCT. In preferred embodiments, the oligonucleotide sequence consists of 3 nucleotides. In more preferred embodiments, the oligonucleotide sequence is GAA.

In certain embodiments, the number of copies of the repeated oligonucleotide sequence lies in a range of values, for example without limitation, 6-1700, 6-34, 35-65, or 66-1700. In preferred embodiments, the trinucleotide repeat GAA is expanded 6-34, 35-65, or 66-1700 times. In more preferred embodiments, the trinucleotide repeat GAA is expanded 66-1700 times.

The polyamides of the invention comprise methylimidazole carboxamides, methylpyrrole carboxamides, aliphatic amino acids, aliphatic desamino amino acids, aliphatic descarboxy amino acids, and chemical modifications thereof. In certain embodiments, the polyamides of the invention comprise linkable units selected from the group consisting of Im, Py, β, and Dp, linked by amide bonds. The term “Im” in a polyamide refers to 1-methyl-1H-imidazole, resulting for example from synthesis using 4-amino-1-methyl-1H-imidazole-2-carboxylic acid, or, in the case of N-terminal Im, 1-methyl-1H-imidazole-2-carboxylic acid, by methods well known in the art The term “Py” refers to 1-methyl-1H-pyrrole, resulting for example from synthesis using 4-amino-1-methyl-1H-pyrrole-2-carboxylic acid, or, in the case of N-terminal Py, 1-methyl-1H-pyrrole-2-carboxylic acid. The term “P” refers to O-alanine (i.e., 3-aminopropanoic acid). The term “Dp” refers to dimethylaminopropylamine. The chemical modifications of polyamides contemplated by the invention include N-alkylation (e.g., N-methyl, N-ethyl, and the like), and covalent attachment of aralkyl, heterocycloalkyl, and chemical probes such as fluorescent dyes (e.g., BODIPY) by chemical synthetic methods well known in the art.

The term “aralkyl” refers to an aryl group bound through an alkylene linkage to the polyamide. The term “aryl” refers to an optionally substituted aromatic ring system, which ring system contains aromatic hydrocarbons such as phenyl or naphthyl, or to a monocyclic aromatic ring structure, a bicyclic aromatic ring structure having 8-10 atoms, or a tricyclic aromatic ring structure having 10-12 atoms, optionally containing one or more, preferably 1-4, more preferably 1-3, and even more preferably 1-2 heteroatoms independently selected from the group consisting of B, O, S, and N, which may be optionally fused with a cycloalkyl or heterocycloalkyl of preferably 5-7, more preferably 5-6 ring members. The term “optionally substituted” refers to independent substitution with 1 to 3 groups or substituents selected from the group consisting of alkyl, cycloalkyl, heterocycloalkyl, halo, hydroxy, alkoxy, acyloxy, aryloxy, cycloalkyloxy, heterocycloalkyloxy, thiol, alkylthio, arylthio, cycloalkylthio, heterocycloalkylthio, alkylsulfinyl, arylsulfinyl, cycloalkylsulfinyl, heterocycloalkylsulfinyl, alkylsulfonyl, arylsulfonyl, cycloalkylsulfonyl, heterocycloalkylsulfonyl, amino, amido, urea, aminosulfonyl, alkylsulfonylamino, arylsulfonylamino, cycloalkylsulfonylamino, heterocycloalkylsulfonylamino, alkylcarbonylamino, arylcarbonylamino, cycloalkylcarbonylamino, heterocycloalkylcarbonylamino, carboxyl, acyl, nitro, and cyano, attached at any available point to produce a stable compound.

The term “alkyl” denotes an optionally substituted alkane-derived radical containing 1-20, preferably 1-15, even more preferably 1-6 carbon atoms, including straight chain and branched chain alkane, having 1-3 optional substitutions as defined in

attached at any available atom to produce a stable compound.

The term “cycloalkyl” denotes an optionally substituted saturated or unsaturated non-aromatic monocyclic, bicyclic or tricyclic carbon ring systems of 3-10, more preferably 3-6, ring members per ring, such as cyclopropyl, cyclopentyl, cyclohexyl, adamantyl, and the like, having 1-3 optional substitutions as defined in [0019] attached at any available atom to produce a stable compound.

The term “heterocycloalkyl” denotes a saturated or unsaturated non-aromatic cycloalkyl group having 5-12 atoms in which from 1 to 3 carbon atoms in the ring are replaced by heteroatoms of B, O, S or N, having optionally fused benzo or heteroaryl of 5-6 ring members, and having 1-3 optional substitutions as defined in [0019] attached at any available atom to produce a stable compound.

The term “halo” denotes all halogens, that is, chloro (Cl), fluoro (F), bromo (Br), or iodo (I).

The term “hydroxyl” denotes the group —OH.

The term “alkoxy” denotes the group —OR^{a}, where R^{a} is alkyl.

The term “acyloxy” denotes the group —OC(O)R^{b}, where R^{b} is hydrogen, alkyl, cycloalkyl, heterocycloalkyl, or aryl.

The term “aryloxy” denotes the group —OR^{c}, where R^{c} is aryl.

The term “cycloalkyloxy” denotes the group —OR^{d}, where R^{d} is cycloalkyl.

The term “heterocycloalkyloxy” denotes the group —OR^{e}, where R^{e} is heterocycloalkyl.

The term “thiol” denotes the group —SH.

The term “alkylthio” denotes the group —SR^{f}, where R^{f} is alkyl.

The term “arylthio” denotes the group —SR^{g}, where R^{g} is aryl.

The term “cycloalkylthio” denotes the group —SR^{h}, where R^{h} is cycloalkyl.

The term “heterocycloalkylthio” denotes the group —SRⁱ, where Rⁱ is heterocycloalkyl.

The term “alkylsulfinyl” denotes the group —S(O)R^{j}, where R^{j} is alkyl.

The term “arylsulfinyl” denotes the group —S(O)R^{k}, where R^{k} is aryl.

The term “cycloalkylsulfinyl” denotes the group —S(O)R^{L}, where R^{L} is cycloalkyl.

The term “heterocycloalkylsulfinyl” denotes the group —S(O)R^{m}, where R^{m} is heterocycloalkyl.

The term “alkylsulfonyl” denotes the group —S(O)₂Rⁿ, where Rⁿ is alkyl.

The term “arylsulfonyl” denotes the group —S(O)₂R^{o}, where R^{o} is aryl.

The term “cycloalkylsulfonyl” denotes the group —S(O)₂R^{p}, where R^{p} is cycloalkyl.

The term “heterocycloalkylsulfonyl” denotes the group —S(O)₂R^{q}, where R^{q} is heterocycloalkyl.

The term “amino” denotes the group —NR^{s}R^{t}, where R^{s} and R^{t} are independently hydrogen or alkyl, or R^{s} and R^{t} together with the nitrogen to which they are attached can form a 5-7 membered heterocycloalkyl or aryl group.

The term “amido” denotes the group —C(O)NR^{u}R^{v}, where R^{u} and R^{v} are independently hydrogen or alkyl, or R^{u} and R^{v} together with the nitrogen to which they are attached form a 5-7 membered heterocycloalkyl or aryl group.

The term “urea” denotes the group —NR^{w}C(O)NR^{x}R^{y}, wherein R^{w} is hydrogen or alkyl, and R^{x} and R^{y} are independently hydrogen or alkyl, or R^{x} and R^{y} together with the nitrogen to which they are attached form a 5-7 membered heterocycloalkyl or aryl group.

The term “aminosulfonyl” denotes the group —S(O)₂NR^{z}R^{aa}, where R^{z} and R^{aa} are independently hydrogen or alkyl, or R^{z} and R^{aa} together with the nitrogen to which they are attached form a 5-7 membered heterocycloalkyl or aryl group.

The term “alkylsulfonylamino” denotes the group —NR^{ab}S(O)₂R^{ac}, wherein R^{ac} is alkyl, and R^{ab} is hydrogen or alkyl.

The term “arylsulfonylamino” denotes the group —NR^{ad}S(O)₂R^{ae}, wherein R^{ae} is aryl, and R^{ad} is hydrogen or alkyl.

The term “cycloalkylsulfonylamino” denotes the group —NR^{af}S(O)₂R^{ag}, wherein R^{ag} is cycloalkyl, and R^{af} is hydrogen or alkyl.

The term “heterocycloalkylsulfonylamino” denotes the group —NR S(O)₂R^{ai}, wherein R^{ai} is heterocycloalkyl, and R^{ah} is hydrogen or alkyl.

The term “alkylcarbonylamino” denotes the group —NR^{aj}C(O)R^{ak}, wherein R^{ak} is alkyl, and R^{aj} is hydrogen or alkyl.

The term “arylcarbonylamino” denotes the group —NR^{al}C(O)R^{am}, wherein R^{am} is aryl, and R^{al} is hydrogen or alkyl.

The term “cycloalkylcarbonylamino” denotes the group —NR^{an}C(O)R^{ao}, wherein R^{ao} is cycloalkyl, and R^{an} is hydrogen or alkyl.

The term “heterocycloalkylcarbonylamino” denotes the group —NR^{ap}C(O)R^{aq}, wherein R^{aq} is heterocycloalkyl, and R^{ap} is hydrogen or alkyl.

The term “carboxyl” denotes the group —C(O)OR^{ar}, where R^{ar} is hydrogen, alkyl, cycloalkyl, heterocycloalkyl, or aryl.

The term “acyl” denotes the group —C(O)R^{as}, where R^{as} is hydrogen, alkyl, cycloalkyl, heterocycloalkyl, or aryl.

In general, polyamides of the present invention may be synthesized by solid phase methods using compounds such as Boc-protected 3-methoxypyrrole, imidazole, pyrrole aromatic amino acids, and alkylated derivatives thereof, which are cleaved from the support by aminolysis, deprotected (e.g., with sodium thiophenoxide), and purified by reverse-phase HPLC, as well known in the art. The identity and purity of the polyamides may be verified using any of a variety of analytical techniques available to one skilled in the art such as ¹H-NMR, analytical HPLC, and/or matrix-assisted laser-desorption ionization time-of-flight mass spectrometry (MALDI-TOF MS-monoisotropic).

Methods useful for the synthesis of polyamides contemplated for use in the practice of the present invention have been reported, for example, in U.S. Pat. Nos. 6,090,947 and 6,555,692, which are hereby incorporated by reference in their entireties and for all purposes.

In the nomenclature of the polyamides of the present invention, the sequence of constituent linkable units is recited, wherein the connecting linkage is an amide linkage. For example, the term “ImPyDp” refers to the chemical structure N-(5-(3-(dimethylamino)propylcarbamoyl)-1-methyl-1H-pyrrol-3-yl)-1-methyl-1H-imidazole-2-carboxamide, with structure of Formula I:

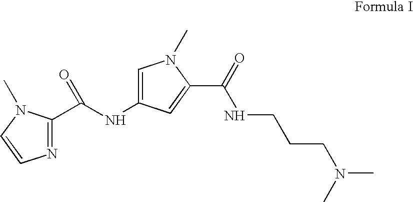

By analogy to a peptide, the Im linkable unit of Formula I is considered to reside at the “N-terminal” of the polyamide, whereas the Dp of Formula I occupies the “C-terminal” position.

It is understood by one of skill in the art that polyamides of the present invention bind double stranded (i.e., duplex) DNA. Accordingly, recitation of a sequence of DNA herein contemplates the recited single-stranded DNA, the complementary (i.e., Watson-Crick) sequence, and the duplex molecule comprising the recited and complementary strands of DNA.

Two classes of polyamides are well established and known in the art: hairpin polyamides which bind mixed sequence DNA with high affinity and specificity (Dervan & Edelson, 2003, Curr. Opin. Struct. Biol. 13:283-299; Trauger et al., 1996, Nature 382:559-561), and linear β-alanine linked polyamides which can target homopurine runs of DNA (Urbach & Dervan, 2001, Proc. Natl. Acad. Sci. U.S.A. 98:4343-4348; Janssen et al., 2000a, Mol. Cell 6:999-1011) such as GAA repeats.

Polyamides able to form hairpin turns bind to predetermined sequences in the minor groove of DNA with affinities and specificities comparable to naturally occurring DNA binding proteins (Trauger et al., Id.; Swalley et al., 1997, J. Am. Chem. Soc. 119:6953-6961; Turner et al., 1997, J. Am. Chem. Soc. 119:7636-7644). Sequence specificity is determined by a code of oriented side-by-side pairings of the polyamides (Wade et al., 1992, J. Am. Chem. Soc. 114, 8783-8794; Mrksich et al., 1992, Proc. Natl. Acad. Sci. U.S.A. 89:7586-7590; Wade et al., 1993, Biochemistry 32, 11385-11389; Mrksich et al., 1993, J. Am. Chem. Soc. 115:2572-2576; White et al., 1997a, Chem. Biol. 4:569-578; White et al., 1997b, J. Am. Chem. Soc. 119:8756-8765). An Im/Py pairing targets a G•C base pair, while a Py/Im pair recognizes C•G. The Py/Py pair is degenerate and targets both AFT and T-A base pairs (Pelton et al., 1989, Proc. Natl. Acad. Sci. USA 86:5723-5727; Chen et al., 1994, Nature Struct. Biol. 1:169-175; White, et al., 1996, Biochemistry 35:12532-12537). The validity of these pairing rules is supported by a variety of polyamide structural motifs which have been characterized by footprinting, affinity cleaving, 2-D NMR, and x-ray methods. As well known in the art, the term “footprinting” refers to a technique for identifying the site on DNA bound by some agent (i.e., protein or polyamide) by virtue of the protection of backbone phosphate DNA bonds against attack by nuclease, or to protection against chemical modification of the DNA bases, afforded by the agent. Polyamides have been found to be cell permeable and to inhibit transcription factor binding and expression of a designated gene (Gottesfeld et al., 1997, Nature 387:202-205).

Linear β-alanine linked polyamides have been shown to bind purine tract sequences in vitro (Urbach & Dervan, Id). and GAGAA (SEQ ID NO:______) repeats in Drosophila satellite DNA both in vitro and in cytological chromosome spreads (Janssen et al., 2000a, Id). These latter molecules induce chromatin opening and reverse heterochromatin-mediated position effect gene silencing when administered to Drosophila embryos (Janssen et al., 2000a, Id.; Janssen et al., 2000b, Mol. Cell 6:101301924). Structural studies indicate that β-alanine linked polyamides bind canonical B-type DNA (Urbach et al., 2002, J. Mol. Biol. 320:55-71). Rules for the recognition of linear polyamide:DNA complexes have been reported (Urbach & Dervan, Id). These rules indicate that an Im linkable unit within a linear polyamide favors all four Watson-Crick duplex DNA basepairs (i.e., A•T, T•A, G•C and C•G), whereas β-alanine and Py favor A•T and T•A basepairs. Without wishing to be bound by theory, given the high affinity of polyamides for their target sites as characterized by dissociation constants of nanomolar to picomolar (Dervan, 2001, Bioorgan. Med. Chem. 9:2215-2235; White et al., 1998, Nature (London) 391:468-471; Turner et al., 1998, J. Am. Chem. Soc. 120:6219-6226), linear β-alanine linked polyamides can be envisaged to act as a thermodynamic “sink” and lock expanded oligonucleotide repeat sequences into a duplex B-type DNA conformation. Such a binding event disfavors duplex unpairing, which is necessary for formation of, for example, triplex and sticky DNA. Alternatively, polyamides may relieve heterochromatin-mediated repression by opening the chromatin domain containing the target gene (e.g., frataxin gene) (Janssen et al. 2000b, Id). Importantly, polyamides bound within coding regions of genes do not necessarily block transcriptional elongation (Gottesfeld et al., 2002, J. Mol. Biol. 121:249-263; Shinohara et al., 2004, J. Am. Chem. Soc. 126:5113-5118; Dickinson et al., 2004, Chem. Biol. 11:1583-1594). Thus, polyamides have the potential to relieve transcription repression at expanded oligonucleotide repeat sequences.

In certain embodiments, the polyamide of the present invention is selected from the group consisting of ImImβImImβImβDp, ImPyβImPyβImβDp, and ImβImImβImImβDp. In preferred embodiments, the polyamide is ImPyβImPyβImβDp.

In certain embodiments, the invention provides a composition comprising a polyamide of the present invention and a pharmaceutically acceptable carrier. The term “composition” refers to a formulation suitable for administration to an intended subject for therapeutic purposes that contains at least one pharmaceutically active compound and at least one pharmaceutically acceptable carrier or excipient. The term “pharmaceutically active” and “therapeutically effective” indicate that the materials or amount of material is effective to prevent, alleviate, or ameliorate one or more symptoms of a disease or medical condition, and/or to prolong the survival of the subject being treated. The term “pharmaceutically acceptable” indicates that the indicated material does not have properties that would cause a reasonably prudent medical or veterinary practitioner to avoid administration of the material to a patient, taking into consideration the disease or conditions to be treated and the respective route of administration. For example, it is commonly required that such a material be essentially sterile, e.g., for injectibles. The term “subject” refers to a mammal, for example, in a veterinary context (e.g., canine, feline) a zoological context (e.g., non-human primate), a commercial context (e.g., bovine, caprine, ovine, porcine, and the like), or to humans. In a preferred embodiment, the subject is a human.

In some embodiments, the composition is a sterile preparation, e.g. for injectibles. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, dextrose solution, and mixed aqueous-organic solution (e.g., DMSO-water). The term “effective amount” of a compound or composition includes a non-toxic but sufficient amount of the particular compound or composition to provide the desired therapeutic effect.

In certain embodiments, the invention provides a method for modulating the transcription of a gene having an expanded oligonucleotide repeat sequence, wherein the method includes contacting the gene with a polyamide which binds the oligonucleotide repeat sequence, and thereby modulates the transcription of the gene. The polyamide is preferably provided at a level sufficient to bind at least 1%, more preferably at least 10%, 20%, 30%, 40%, 50%, 60%, 70%, 80%, 90%, 99%, or even higher, of the expanded oligonucleotide sequence. In certain embodiments, the compound will be at a concentration of about 0.1 nM, 1 nM, 100 nM, 1 μM, 10 μM, 100 μM, 1 mM, or in a range of 0.1-10 nM, 10-100 nM, 100-500 nM, 500-1000 nM, 1-100 μM, 100-500 μM, or 500-1000 μM. It is understood that the recitation of ranges of values herein contemplates additional ranges described by taking any two different endpoints from the ranges of values as the endpoints of the additional ranges; e.g., the range 0.1 nm to 1000 μM is contemplated by the ranges of values immediately above. The term “about” in the context of a numeric value refers to the numeric value +/−10%. In certain embodiments, the modulation is an increase in transcription. In other embodiments, the modulation is a decrease in transcription.

In certain embodiments, the target gene for the invention method of modulation of transcription is DRPLA, HD, AR, ATXN1, ATXN2, ATXN3, CACNA1A, ATNX7, FMR1, FMR2, FXN, DMPK, SCA8, SCA10, SCA12, or ZNF9. In preferred embodiments, the gene is FXN. In certain embodiments, the oligonucleotide sequence which is expanded in the target gene contains 3, 4, or 5 nucleotides. In certain embodiments, the expanded oligonucleotide sequence is CGG, GCC, GAA, CTG, CAG, CCTG, or ATTCT. In preferred embodiments, the expanded oligonucleotide sequence consists of 3 nucleotides, more preferably the sequence GAA. In certain embodiments, the number of repeats in the oligonucleotide expansion is in the range 6-1700, 6-34, 35-65, or 66-1700, more preferably 35-65 or 66-1700.

In preferred embodiments, the target gene resides within a cell. In further preferred embodiments, the polyamide of the invention localizes in the nucleus of the cell. In further embodiments, such localization is monitored. Nuclear localization is conveniently monitored by, for example, fluorescence microscopy of fluorescent labeled polyamide by methods well known in the art. Additional methods of monitoring include deconvolution and phase contrast microscopy and radioautography, all well known in the art.

In certain embodiments, the polyamide used in the invention method of modulating the transcription of a gene having expanded oligonucleotide repeat sequences includes the linkable units Im, Py, β, Dp, and chemical derivatives thereof. In certain embodiments, the linkable units include Im, Py, β, Dp, and desamino, descarboxy, N-alkyl (i.e., N-methyl), and covalently attached aralkyl and heterocycloalkyl derivatives thereof. In preferred embodiments, the linkable units include Im, Py, β, Dp, and desamino, descarboxy, N-alkyl (e.g., N-methyl), aralkyl and heterocycloalkyl derivatives thereof, and derivatives thereof conjugated with chemical probes. In further preferred embodiments, the linkable units include Im, Py, β, Dp, and desamino, descarboxy, and N-alkyl (i.e., N-methyl) derivatives thereof. In more preferred embodiments, the linkable units include Im, Py, β, and Dp. In certain embodiments, the polyamide is ImImβImImβImβDp, ImPyβImPyβImβDp, ImβImImβImImβDp, or derivatives thereof conjugated with chemical probes. In certain embodiments, the polyamide is ImImβImImβImβDp, ImPyβImPyβImβDp, or ImβImImβImImβDp. In preferred embodiments, the polyamide is ImPyβImPyβImβDp.

In certain embodiments, the invention provides methods for treating a subject suffering from a disease associated with a gene having an expansion of an oligonucleotide repeat sequence (e.g., trinucleotide repeat disorders) in which treatment is effected by administering to the subject a therapeutically effective amount of a polyamide which binds the oligonucleotide sequence and thereby modulates transcription of the gene. In preferred embodiments, the subject is a human. The polyamides of the invention may be administered by different routes including intravenous, intraperitoneal, subcutaneous, intramuscular, oral, transmucosal, rectal, or transdermal, as well known in the art.

In certain embodiments, the disease (i.e., target disease) suffered by the subject is dentatorubropallidoluysian atrophy, Huntington's disease, spinobulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 10, spinocerebellar ataxia type 12, fragile X syndrome, fragile XE syndrome, Friedreich's ataxia, and myotonic dystrophy type 1, or myotonic dystrophy type 2.

In preferred embodiments, the disease is Friedreich's ataxia (i.e., FRDA). The neurodegenerative disease Friedreich's ataxia is caused by hyper-expansion of GAA repeats in the first intron of a nuclear gene that encodes the essential mitochondrial protein frataxin (Campuzano et al., 1996, Science 271:1423-1427; Montermini et al., 1997, Ann. Neurol. 41:675-682; Wells et al., 1998, Genetic Instabilities and Hereditary Neurological Diseases, Academic Press, San Diego, Calif.; Pandolfo, M., 2003, Semin. Pediatr. Neurol. 10:163-172). The term “frataxin” refers to a protein encoded by five exons that span a 40 kilobase region of chromosome 9q13-q21.1, which gene is customarily accorded the name FXN; surrogate names for this gene include “FRDA” and “X25.” The term “FRDA” in the context of a disease refers to Friedreich's ataxia, whereas in the context of a gene, FRDA refers to the FXN gene. Normal frataxin alleles have an expansion of 6-34 GAA repeats while FRDA patient alleles have hyper-expansion of 66-1700, or even more, repeats. GAA repeats interfere with gene transcription (Ohshima et al., 1998, J. Biol. Chem. 273:14588-14595; Bidichandani et al., 1998, Am. J. Hum. Genet. 62:111-121; Grabczyk & Usdin, 2000a, Nucleic Acids Res. 28:2815-2822) and longer repeats cause a more profound frataxin deficiency and are associated with earlier onset and increased severity of the disease (Pandolfo, Id). Biochemical studies have documented that expanded GAA repeats adopt unusual non-B type DNA structures such as triplexes containing two purine GAA strands along with one pyrimidine TTC strand, flanking a single-stranded pyrimidine region (Oshima, Id.; Sakamoto et al., 1999, Mol. Cell 3:465-475), as well as intramolecular “sticky” DNA (Bidichandani et al., Id.; Vetcher et al., 2002a, J. Biol. Chem. 277:39228-39234; Vetcher et al., 2002b, J. Biol. Chem. 277:39217-39227; Napierala et al., 2004, J. Biol. Chem. 279:6444-6454; Vetcher & Wells, 2004, J. Biol. Chem. 279:6434-6443). As known in the art, long duplex (i.e., GAA•TTC) repeat sequences form “sticky” DNA characterized by two separate repeating tracts associated within a single closed plasmid DNA. The interaction of the two tracts requires the repeats be oriented in the direct repeat orientation, negative supercoiling, and the presence of divalent metal ions to stabilize the DNA-DNA associated region (Sakamoto et al., 1999, Id.; Vetcher et al., 2002a, Id.; Vetcher et al., 2002b, Id). It has been demonstrated that sticky DNA has the capacity to form both in vitro (Vetcher et al., 2002b, Id). and in vivo (Napierala et al., Id.; Vetcher & Wells, Id). Triplexes and/or sticky DNA block elongation by RNA polymerase II (Ohshima et al., Id). Other studies indicate that expanded duplex GAA•TTC repeats induce repressive heterochromatin when introduced into reporter genes in vivo (Saveliev et al., 2003, Nature 422:909-913).

Without wishing to be bound by theory, the present invention provides a novel therapeutic approach for treatment of a disease (e.g., Friedreich's ataxia) based on alleviating transcription repression of a gene (e.g., frataxin gene) with small sequence-specific DNA ligands (i.e., polyamides). For example, molecules that interfere with triplex/sticky DNA and/or heterochromatin formation in the frataxin gene can increase successful elongation (i.e., transcription) through expanded GAA repeats, thereby relieving the deficiency in frataxin mRNA and protein in FRDA affected individuals (Napierala et al., Id.; Saveliev et al., Id.; Grabczyk & Usdin, 2000b, Nucleic Acids Res. 28:4930-4937). Similarly, other diseases which are caused by transcriptional repression due to expanded oligonucleotide repeat sequence can be treated by methods of the present invention. Among the cell permeable small molecules that bind DNA (Gottesfeld et al., 2000, Gene Expr. 9:77-91), the pyrrole-imidazole polyamides of the present invention are unique in that they can be synthesized so as to recognize predetermined DNA sequences through an amino acid-base recognition code (Dervan & Edelson, Id). Cell permeation can be monitored by methods well known in the art, including microscopic techniques using chemical probe conjugated polyamides discussed herein. Additionally, cell permeation can be monitored by measurement of product mRNA (see Example 8) and product protein (see Examples 3-4).

In certain embodiments, the gene associated with a target disease to which the polyamide of the invention binds is DRPLA, HD, AR, ATXN1, ATXN2, ATXN3, CACNA1A, ATNX7, FMR1, FMR2, FXN, DMPK, SCA8, SCA10, SCA12, NTR, or ZNF9. In preferred embodiments, the gene is FXN. In certain embodiments, the gene associated with a target disease includes an expanded oligonucleotide sequence which consists of 3, 4, or 5 nucleotides. Examples of oligonucleotide sequences contemplated by this aspect of the invention include, without limitation, CGG, GCC, GAA, CTG, CAG, CCTG, and ATTCT. In some embodiments, the oligonucleotide sequences include, without limitation, CGG, GCC, GAA, CTG, and CAG. In other embodiments, the oligonucleotide sequence is, without limitation, CCTG. In further embodiments, the oligonucleotide sequence is, without limitation, ATTCT. In preferred embodiments, the oligonucleotide sequence consists of 3 nucleotides. In more preferred embodiments, the oligonucleotide sequence is GAA. In certain embodiments, the number of copies of the expanded oligonucleotide sequence lies in a range of values, for example without limitation, 6-1700, 6-34, 35-65, 66-1700. In preferred embodiments, the trinucleotide repeat GAA is expanded 6-34, 35-65, or 66-1700 times. In more preferred embodiments, the trinucleotide repeat GAA is expanded 66-1700 times.

In certain embodiments, the polyamide used in the method directed to treating a subject suffering from a disease associated with a target gene having expanded oligonucleotide repeat sequences includes the linkable units Im, Py, β, Dp, and chemical derivatives thereof. In certain embodiments, the linkable units include Im, Py, β, Dp, and desamino, descarboxy, N-alkyl (i.e., N-methyl), aralkyl, heterocycloalkyl, and chemical probe conjugated derivatives thereof. In preferred embodiments, the linkable units include Im, Py, β, Dp, and desamino, descarboxy, and N-alkyl (i.e., N-methyl) derivatives thereof. In more preferred embodiments, the linkable units include Im, Py, β, and Dp. In certain embodiments, the polyamide is ImImβImImβImβDp, ImPyβImPyβImβDp, ImβImImβImImβDp, or derivatives chemical probe conjugated thereof. In certain embodiments, the polyamide is ImImβImImβImβDp, ImPyβImPyβImβDp, or ImβImImβImImβDp. In preferred embodiments, the polyamide is ImPyβImPyβImβDp. In certain embodiments, modulation of transcription of the target gene is an increase in transcription. In other embodiments, modulation is a decrease in transcription.

### EXAMPLES

### Example 1

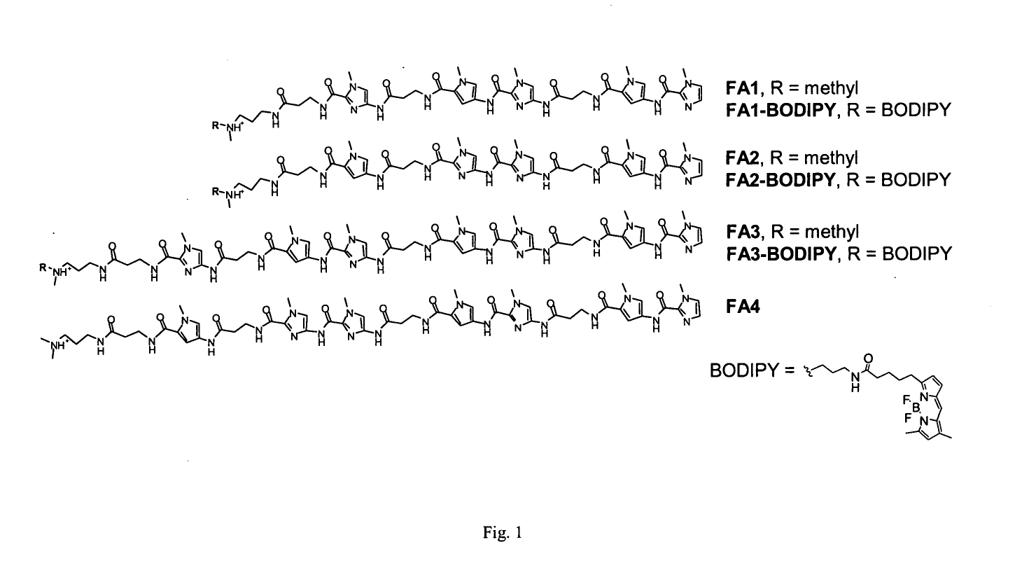

Targeting GAA Repeat DNA with Polyamides. β-Alanine linked polyamides FA1-FA6 were synthesized (see Example 6) with sequence, theoretical DNA binding site, theoretical DNA binding site SEQ ID NO: ______) and binding affinity as shown in Table 1. The chemical structure of FA1-FA4, BODIPY, and BODIPY-conjugated FA1-FA4 is provided in FIG. 1. Quantitative DNase I footprinting (Trauger & Dervan, 2001, Methods Enzymol. 340:450-466) demonstrated that FA1 bound to a radiolabeled PCR product containing a (GAA)₆ (SEQ ID NO:______) sequence with an apparent dissociation constant (K_{D}) of 0.1 nM (Table 1). FA3 exhibits a K_{D} of ˜3 pM in footprinting experiments performed at low (i.e., ˜2 pM) DNA concentrations (Table 1). Those of skill in the art will recognize that this value may be an underestimation of the affinity of this molecule for GAA repeat DNA since the K_{D} measurements are limited by a minimum DNA concentration of ˜2 pM in the binding reaction. Radiolabeling of nucleic acid incorporated γ³²P ATP and polynucleotide kinase used standard procedures.

TABLE 1
Polyamides designed to target GAA · TTC
duplex repeats in the frataxin gene.

| | (GAA)ₙ repeat | Target | |
|---|---|---|---|
| | number in | site | Binding |
| | target site, | SEQ ID | affinity |
| Polyamide sequence | n = | NO: | (K_{D}, nM)¹ |
| FA1: | 3 | — | 0.11 ± 0.02 |
| ImPyβImPyβImβDp² | | | |
| FA2: | 3 | — | >100 |
| ImPyβImImβPyβDp³ | | | |
| FA3: | 4 | — | 0.003 ± 0.001 |
| (ImPyβ)₃ImβDp | | | |
| FA4: | 4 | — | 2.0 ± 0.4 |
| (ImPyβ)₂ImImβPyβDp³ | | | |
| FA5: | 5 | — | 0.20 ± 0.02 |
| (ImPyβ)₄ImβDp | | | |
| FA6: | 6 | — | 0.22 ± 0.08 |
| (ImPyβ)₅ImβDp | | | |

¹Binding affinities (mean values of the K_{D} from a minimum of two determinations, and standard deviations) determined by quantitative DNase I footprinting.
²Im, imidazole; Py, pyrrole; β, β-alanine; Dp, dimethylaminopropylamide.
³Mismatch amino acids are underlined.

Changing the sequence of Im and Py amino acids in FA1, without changing the chemical composition of the molecule (i.e., FA2, ImPyβImImβPyβDp, Table 1), reduces the binding affinity by greater than three orders of magnitude (K_{D}=>100 nM, Table 1). Similarly, changing the sequence of FA3 to yield FA4 (ImPyβImPyβImImβPyβDp, Table 1), results in binding affinity reduction by three orders of magnitude to 2 nM, compared to FA3 (Table 1). FA1 is also able to bind extended regions of duplex DNA having GAA repeats ((GAA)₃₃, SEQ ID NO ______) with no loss in affinity, with several molecules of FA1 bound per DNA molecule. In this experiment, 50% occupancy of the DNA is observed at the target site concentration in the reaction (˜1.5 nM), as expected based on the high affinity of the polyamide for GAA•TTC repeat duplex DNA. The term “mismatch” in the context of polyamides or linkable units refers to replacement of one or more linkable units in a polyamide such that recognition for a specific duplex DNA pair is energetically less favorable (Dervan & Edelson, Id.; Urbach & Dervan, Id). The term “match” in the context of polyamide binding to DNA refers to a linkable unit selected according to the polyamide recognition rules of Urbach & Dervan (Id). or Dervan & Edelson (Id).

Two additional molecules, FA5 [(ImPyβ)₄ImβDp] and FA6 [(ImPyβ)₅ImβDp], were synthesized in order to target longer regions of GAA repeats in duplex DNA; i.e., (AAG)₅ (SEQ ID NO: ______) and (AAG)₆ (SEQ ID NO: ______) respectively. These polyamides have binding affinities for duplex GAA•TTC repeat DNA comparable to that of FA1 (Table 1), but are less specific than FA1 or FA3, yielding non-specific binding at high polyamide concentrations. The polyamide ImPyβImβDp targeting the six bp sequence 5′-AAGAAG-3′ (SEQ ID NO: ______) exhibited a low μM binding affinity. As another test for sequence specificity, footprinting experiments with FA1 and FA3 and a radiolabeled DNA fragment containing a mismatch DNA sequence 5′-GGAGGAGGTGGAGGAGGA-3′ (SEQ ID NO: ______) were performed. Neither FA1 nor FA3 bound this DNA sequence at polyamide concentrations up to 100 nM.

### Example 2

Nuclear Localization of Fluorescent Polyamides. BODIPY-conjugated fluorescent derivatives of the match polyamides FA1 and FA3 and mismatch polyamide FA2 were synthesized, with the dye attached at the carboxyl terminus of the polyamide (FIG. 1). Quantitative DNase I footprinting demonstrated that polyamides FA1- and FA3-BODIPY retain the full sequence specificity of the parent polyamides but exhibit 13- to 20-fold losses in binding affinity for (GAA)₆ DNA (SEQ ID NO: ______), compared to the unconjugated polyamides; for FA1-BODIPY, K_{D}=1.3 nM; for FA3-BODIPY, K_{D}=0.04 nM.

Epstein Barr virus-transformed lymphoblast cell lines from an FRDA patient (line GM15850) and from his/her unaffected sibling (line GM15851) were obtained from the NIGMS Human Genetic Cell Repository (Coriell Institute, Camden, N.J.). Both the match FA1-BODIPY and mismatch FA2-BODIPY conjugates localize in the nucleus of live, unfixed normal and FRDA lymphoid cells after 16 h incubation in culture medium, as determined by deconvolution microscopy. The BODIPY-conjugates of the longer polyamides FA3, FA5, and FA6 also localize in the nucleus of the FRDA cells. The degree of nuclear fluorescence observed with these polyamide-BODIPY conjugates suggests that these molecules are binding to numerous sites in human DNA, consistent with the high frequency of occurrence of short (GAA)ₙ(n=<6) repeats in human non-coding repetitive DNA elements (Clark et al., 2004, Genomics 83: 373-383).

### Example 3

GAA Specific Polyamides Up Regulate Frataxin mRNA and Protein. To assess whether polyamides alleviate transcription inhibition caused by expanded GAA repeats in the frataxin gene, quantitative real time/reverse transcriptase PCR (qRT-PCR) was used to monitor frataxin mRNA levels in the GM15850 and GM15851 lymphoid cell lines described above; levels of glyceraldehyde 3-phosphate dehydrogenase (GAPDH) mRNA were used as an internal control for each RNA sample; see Example 8. No differences in GAPDH were found between the two cell lines. The FRDA cell line had a markedly lower level (i.e., 6-13%, range of >50 determinations) of frataxin mRNA compared to the cell line from the normal individual. The FRDA and control cells were incubated with various concentrations of each of the polyamides for various lengths of time and found that only polyamide FA1 increased frataxin mRNA levels after 7 days incubation in culture medium. No changes in frataxin mRNA levels were observed on shorter incubation times. Over the concentration range of 1 to 8 μM, the tested polyamides were not cytotoxic to the lymphoid cell lines, as determined by trypan blue exclusion and measurements of ATP levels as conducted by standard protocols known to one of skill in the art, and do not affect cell growth rates.

The level of frataxin mRNA in the FRDA GM15850 cell line was increased 2.5-fold by incubation with polyamide FA1 at 2 μM. The range of fold increases observed with 2 μM FA1 in the FRDA cell line in numerous experiments is 2.0 to 3.6, resulting in as much as 45% of the level of frataxin mRNA found in the normal cell line. Neither higher concentrations of FA1 nor longer incubations times increased frataxin transcription above the levels observed at 2 μM upon 7-day incubation. Polyamide FA1 did not change frataxin mRNA levels in the cell line derived from the normal individual. Similar incubations with the mismatch polyamide FA2 caused a modest increase in frataxin transcription in the FRDA cell line but no change in frataxin mRNA in the normal cell line. The levels of GAPDH mRNA were not changed by polyamide treatment in either cell line. Since mildly affected FRDA individuals have between 25-40% of the frataxin mRNA levels of homozygous normal individuals, and heterozygous carrier non-affected individuals have ˜40-50% of normal levels, a potentially therapeutic increase in frataxin mRNA is afforded by incubation with the polyamide of the invention. Since the GAA repeats in the FXN gene are within an intron, and hence do not affect the sequence of the frataxin protein, such an increase in frataxin mRNA would be of therapeutic benefit.

Experiments were conducted to determine the effect of removal of polyamide FA1 from the culture medium on frataxin transcription. After induction of frataxin mRNA synthesis by FA1 (7 days at 2 ƒM), transfer of the cells to fresh medium lacking polyamide caused frataxin mRNA levels to decrease to pre-treatment levels after 96 h. Thus, polyamides must be continuously present to maintain active transcription of FRDA frataxin alleles. Without wishing to be bound by theory, the finding that incubation periods of 7 days or more are necessary to give rise to observable increases in frataxin mRNA suggests that multiple rounds of DNA replication are necessary for polyamide to alter either the DNA or chromatin structure of expanded frataxin alleles, leading to active transcription, and that removal of polyamide caused the frataxin gene to re-adopt its inactive DNA or chromatin conformation.

Interestingly, the highest affinity compound, FA3, did not increase frataxin mRNA levels, whereas the fluorescent (i.e., BODIPY-conjugated) version of this molecule localized in the nucleus in the FRDA lymphoid cells. Previous studies have established that nuclear localization is very sensitive to polyamide composition and structure, and especially the nature of the carboxyl-terminus (Best et al., Id.; Edelson et al., 2004, Nucleic Acids Res. 32:2802-2818); therefore, the non-fluorescent version of FA3 may not enter the nucleus. To test this hypothesis, the levels of frataxin mRNA were monitored after incubation with FA3-BODIPY, which revealed a ˜2-3-fold increase in relative levels of frataxin mRNA (compared to GAPDH) after 2-4 day incubations. One skilled in the art will appreciate that modulation of the hydrophobic character of the polyamide, for example via attachment of fluorescent dye (e.g., BODIPY), aralkyl and heterocycloalkyl functionalities at the C-terminal of the polyamide, can modulate nuclear localization of the polyamide.

Since the primary transcripts from FRDA frataxin genes contain long stretches of GAA repeat RNA sequence, it is conceivable that this RNA will not be correctly processed into mature frataxin mRNA and frataxin protein will not be produced. To test whether polyamide FA 1 leads to increased levels of frataxin protein in treated lymphoid cells, total cell extracts from polyamide-treated (1-2 μM for 7 days) and untreated GM15851 control and GM15850 FRDA cells were subjected to SDS-PAGE, and the corresponding blots were probed with anti-frataxin or anti-actin antibodies. SDS-PAGE (i.e., sodium dodecylsulfate-polyacrylamide gel electrophoresis) was conducted by standard methods well known in the art. A ˜2-3-fold increase in frataxin protein was observed with FA1 in the FRDA cells, which correlated with the observed increase in frataxin mRNA.

### Example 4

Effects of Polyamides on Global Gene Expression. DNA microarray analyses (see Example 10) were performed with RNA isolated from GM15850 FRDA and GM15851 normal lymphoid cells that were either untreated or treated with polyamides FA1 (at 1 and 2 μM) or FA2 (at 2 μM) for 7 days on Affymetrix Human Genome U133 Plus 2.0 GeneChips®. These chips contain ˜106 oligonucleotides representing all or nearly all of the genes in the human genome). Experiments were conducted according to manufacturer guidelines. FA1 was found to affect the mRNA levels for a limited number of genes in the FRDA cell line (at P≦0.005, 51 genes affected by 1 μM FA1, and 16 genes affected by 2 μM FA1), and only two genes in the normal cell line. Although more genes were judged affected by FA1 at 1 μM than at 2 μM, this difference is largely due to genes whose mRNA levels change by less than −25% in either direction, and thus are not highly significant. At 2 μM FA1, 15 genes were increased in expression by greater than 50% and one gene was decreased by 45%. At 1 μM FA1, only three genes had comparable changes in their mRNA levels. For GM15851 cells, two genes were up regulated by FA1, and no genes were down regulated. (Note that the probability that the genes affected in GM15851 cells would be judged significant by chance at P≦0.005 is 0.8-1.0, based on analysis with BRB ArrayTools). For the frataxin gene, untreated GM15850 cells showed 16.7% of the frataxin mRNA found in untreated GM15851 cells, and incubation with FA1 at 2 μM increases frataxin mRNA by 2.5-fold, bringing the frataxin mRNA level in GM15850 cells to 42% of that found in GM15851 cells. These values are comparable to those obtained by qRT-PCR. Strikingly, binding sites for FA1 (5′-AAGAAGAAG-3′, SEQ ID NO: ______) are present in 18/20 genes whose mRNA levels are increased by 1.2-fold or greater in GM15850 cells, although this sequence would be expected to appear at random only once per 262,000 bp in genomic DNA.

Transcript levels for frataxin were not changed by FA2 in either cell line, and FA2 affected only a small number of genes in either cell line; at P≦:0.005, three genes were affected by 2 μM FA2 in GM15850 cells and one gene was affected in GM15851 cells, which could have been called by chance.

To examine the overall changes in gene expression profiles in treated and untreated populations of FRDA and control cells, the differences in the geometric mean of the expression signals between each of the experimental conditions were plotted and examined. At a P value of ≦:0.005, a total of 632 genes were judged significant between all conditions. Neither the match FA1 or mismatch FA2 polyamide affected the profile of GM15851 cells (slope of the correlation between conditions=−0.04 to +0.04), whereas FA1-treated GM15850 FRDA cells (at 2 μM) have a gene expression profile that approaches that of untreated GM15851 cells compared to untreated FRDA cells (slope=0.69 compared to 0.97). This effect is seen to a lesser degree at 1 μM FA1, but was not seen with the mismatch polyamide FA2. These changes in gene expression in the affected cell line may be a consequence of changes in frataxin protein levels, or some could be direct effects of the polyamide, as suggested by the occurrence of FA1 binding sites in up regulated genes. Taken together, these data are consistent with polyamide FA1 increasing frataxin gene expression and perhaps downstream targets of frataxin, but this molecule has a highly limited effect on global gene expression, suggesting that GAA•TTC-specific polyamides should not be toxic due to aberrant gene expression effects. This hypothesis is supported by the observations that GAA•TTC-specific polyamides have no effects on lymphoid cell morphology, metabolism or growth in culture. Moreover, a search of GenBank reveals that most regions of GAA•TTC DNA sequence [(GAA•TTC)₆ or longer] are present in non-transcribed repetitive DNA elements in the human genome (Clark et al., Id).

### Example 5

Influence of Polyamides on Sticky DNA Conformation. Plasmid pRW4886, which contains two tracts of (GAA)₁₇₆ (SEQ ID NO: ______) in a direct repeat orientation (Vetcher et al., 2002b, Id)., and forms sticky DNA, was treated with polyamides at concentrations of 0-50 μM at 37° C. for 1 hr. Restriction digestion with EcoNI following the polyamide incubation enabled visualization of the presence or absence of an EcoNI cleaved sticky DNA band that runs with decreased mobility compared to the linearized plasmid on 1% agarose gels. Quantitation was by densitometric analysis using Fluor Chem version 3.04 (Alpha Innotech Corp).

The capacity of sequence-specific polyamides to disrupt the intramolecular sticky DNA structure formed by GAA repeat tracts was investigated. Plasmids harboring the sticky DNA structure are visualized by gel electrophoresis after restriction endonuclease cleavage, by methods well known in the art. Linear DNA is indicative of disruption of the sticky DNA structure by a polyamide, whereas the cleaved sticky DNA band that migrates with a much slower mobility reveals no influence of polyamide. Plasmid pRW4886, which contains two tracts of (GAA)₁₇₆ (SEQ ID NO: ______) in a direct repeat orientation (Vetcher et al., 2002b, Id.), was incubated with each of polyamides FA1, FA2, FA3 and FA4 at concentrations ranging from 0-50 μM. The polyamide-bound DNA was then digested with EcoNI and subjected to electrophoresis on 1% agarose gels to determine the amount of EcoNI cleaved sticky DNA retarded band present. Incubation of pRW4886 DNA with FA3 shifted the equilibrium from a maximum amount of sticky DNA to a complete loss of the EcoNI cleaved sticky DNA retarded band at a concentration of 50 nM. FA4, which is a mismatch of FA3 and has a binding affinity ˜1000-times less than FA3 (Table 1), did not affect the stability of sticky DNA in pRW4886 below an FA4 concentration of 5 μM. For FA1, a 1 μM concentration was needed to dissociate the DNA-DNA structure-forming region. The mismatched polyamide FA2, having the lowest binding affinity of all of the polyamides tested, showed no effect on sticky DNA stability even at 100 μM concentration. Thus, the binding affinities of the polyamides for the GAA sequence had an intimate relationship with the concentration needed to shift the equilibrium from the DNA•DNA associated structure to the duplex conformation. The absence of an EcoNI cleaved sticky DNA retarded band demonstrates the capacity of the sequence-specific polyamide binding to shift the non-B to B-DNA equilibrium towards a conventional DNA duplex conformation in supercoiled plasmids. Since sticky DNA inhibits transcription (Ohshima et al., Id.; Sakamoto et al., 2001, J. Biol. Chem. 276:27171-27177), and since the polyamides destabilize this conformation by shifting the structural equilibrium to duplex B-type DNA, it is highly likely that the increases in frataxin mRNA observed with polyamide FA1 are due to this structural transition.

### Example 6

Polyamlde Synthesis and Characterization. Polyamides were synthesized by solid phase methods (Urbach & Dervan, Id.; Baird & Dervan, 1996, J. Am. Chem. Soc. 118:6141-6146) and their identity and purity verified by MALDI-TOF MS and analytical HPLC, using methods well known in the art. Fluorescent conjugates were prepared by coupling BODIPY (Molecular Probes) to the carboxyl-terminus (Best et al., 2003, Proc. Natl. Acad. Sci. U.S.A. 100:12063-12068). Binding affinities for match and mismatch sites were determined by quantitative DNase I footprinting (Trauger & Dervan, Id). A plasmid harboring six GAA repeats was constructed by cloning the oligonucleotide

| | |
|---|---|
| 5′-GCCTTACGGTTACACTTGATGAAGA | (SEQ ID NO:_) |
| AGAAGAAGAAGAATTCGCAATGCCATTG | |
| CGCTATGA-3′ | |

in the pCR2.1 TOPO vector (Invitrogen, Calif.), and a 117 bp singly end-labeled PCR product was generated from this plasmid with the following oligonucleotides:

| | |
|---|---|
| 5′-GTACCTACTAGTCCAGTGTGG-3′ | (SEQ ID NO:_) |
| and | |
| 5′-CTCGATATCTGCAGAATTGCC-3′, | (SEQ ID NO:_) |

where the second oligonucleotide was labeled with γ-³²P ATP and polynucleotide kinase, using standard procedures, to generate a PCR product labeled on the GAA strand. A 204 bp singly end-labeled PCR product was derived from plasmid pMP142 DNA, containing 33 GAA repeats (Ohshima et al., Id.), with following oligonucleotides:

| | |
|---|---|
| 5′-GGCCAACATGGTGAAACC-3′ | (SEQ ID NO:_) |
| and | |
| 5′-GTAGCTGGGATTACAGGCGC-3′. | (SEQ ID NO:_) |

The first oligonucleotide shown was radiolabeled as above to generate a PCR product labeled on the GAA strand. A 150 bp PCR product containing a (GGA•TCC)₆ mismatch sequence was derived from the erbB2 (Her2-neu) promoter in human genomic DNA with the following oligonucleotides:

| | |
|---|---|
| 5′-CTTGTTGGAATGCAGTTGGA-3′ | (SEQ ID NO:_) |
| and | |
| 5′-GGTTTCTCCGGTCCCAAT-3′, | (SEQ ID NO:_) |

with the first oligonucleotide radiolabeled by standard procedures.

### Example 7

Cell Culture. Epstein Barr virus transformed lymphoblast cell lines GM15850 from a FRDA patient (alleles with 650 and 1030 GAA repeats in the frataxin gene, from the Coriell Cell Repository, Camden, N.J.), and GM15851 from an unaffected sibling (normal range of repeats), were propagated in RMPI 1640 medium with 2 mM L-glutamine and 15% fetal bovine serum at 37° C. in 5% CO₂. Cell growth and morphology were monitored by phase contrast microscopy and viability by trypan blue exclusion and an ATP assay (ApoSENSOR™, BioVision). Polyamides were added directly to the culture medium in PBS. Nuclear localization of the polyamides was verified by deconvolution microscopy, by methods well known in the art (see e.g., Dudouet et al., Chem. Biol., 2003, 10: 859-867).

### Example 8

Real-Time Quantitative RT-PCR. Real-time quantitative RT-PCR (qRT-PCR) analysis was performed essentially as previously described (Chuma, et al., 2003, Hepatology 37:198-207), using the following primers for the frataxin gene:

| | |
|---|---|
| 5′-CAGAGGAAACGCTGGACTCT-3′ | (SEQ ID NO:_) |
| and | |
| 5′-AGCCAGATTTGCTTGTTTGG-3′. | (SEQ ID NO:_) |

RNA was standardized by quantification of GAPDH mRNA (Pattyn et al., 2003, Nucleic Acids Res. 31:122-123), and all values are expressed relative to GAPDH. qRT-PCR was performed using iScript One-Step RT-PCR kit with SYBR green (Biorad). Statistical analysis was performed on three independent quantitative RT-PCR experiments for each RNA sample.

### Example 9

Western Blot Analysis. Total cell extracts were used for SDS-PAGE and western blotting with antibodies to human frataxin (Chemicon) or actin (Santa Cruz Biotechnology) as a control for cell number and protein loading. Signals were detected by chemiluminescence after probing the blot with HRP-conjugated secondary antibody (Supersignal West, Pierce). To quantify the relative levels of proteins, autoradiograms (within the linear response range of X-ray film) were converted into digital images and the signals quantified using Molecular Dynamics ImageQuant software.

### Example 10

DNA Microarrays. FRDA and control lymphoid cells (i.e., lines GM15850 and GM15851, respectively) were incubated with polyamides FA1 (at 1 or 2 μM) or FA2 (at 2 μM), or in the absence of polyamide, in triplicate for seven days prior to RNA purification and microarray analysis. Affymetrix U133A Plus 2.0 GeneChips® were hybridized in groups of eight for each of the three replicates. Raw GeneChip® data were normalized with RMAExpress (Bolstad et al., 2003, Bioinformatics 19:185-193) and the normalized data was filtered to remove probesets called absent on 24 of 24 chips from class comparisons. The Affymetrix probeset-level data was imported to BRB Arraytools (v3.3.0 Beta 3a) selecting the U133 chips used in the experiment and leaving all filters off. For class comparisons between groups of arrays, unpaired samples were used and the random variance model was selected, with the univariate significance threshold set to 0.005. The restrictions for the univariate test were maintained as the default values of 10 for the maximum number of false discovered genes, 0.1 for the maximum proportion of false discoveries, and a 90% confidence level. Because of poor data correlation in one set of replicates, class comparisons were performed using all chips for the control group versus two of the three replicates for the treatment group (i.e., five groups are the minimum number required for class comparisons).

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to provide additional polyamides and/or various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any two different values as the endpoints of a range. Such ranges are also within the scope of the described invention.

Thus, additional embodiments are within the scope of the invention and within the following claims.

SEQ ID NOs:

| | |
|---|---|
| (GAA)₃ | (SEQ ID NO:_) |
| (GAA)₄ | (SEQ ID NO:_) |
| (GAA)₅ | (SEQ ID NO:_) |
| (GAA)₆ | (SEQ ID NO:_) |
| (GAA)₃₃ | (SEQ ID NO:_) |
| (GAA)₁₇₆ | (SEQ ID NO:_) |
| (AAG)₂ | (SEQ ID NO:_) |
| (AAG)₃ | (SEQ ID NO:_) |
| (AAG)₅ | (SEQ ID NO:_) |
| (AAG)₆ | (SEQ ID NO:_) |
| AGGAGGAGGTGGAGGAGGA | (SEQ ID NO:_) |
| GCCTTACGGTTACACTTGATGAAGAAGAAGAAGAAGA | (SEQ ID NO:_) |
| ATTCGCAATGCCATTGCGCTATGA | |
| GTACCTACTAGTCCAGTGTGG-3′ | (SEQ ID NO:_) |
| CTCGATATCTGCAGAATTGCC-3′ | (SEQ ID NO:_) |
| GGCCAACATGGTGAAACC-3′ | (SEQ ID NO:_) |
| GTAGCTGGGATTACAGGCGC-3′ | (SEQ ID NO:_) |
| 5′-CTTGTTGGAATGCAGTTGGA-3′ | (SEQ ID NO:_) |
| 5′-GGTTTCTCCGGTCCCAAT-3′ | (SEQ ID NO:_) |

What is claimed is:

1. A method for modulating the transcription of a gene, wherein said gene comprises a plurality of repeats of an oligonucleotide sequence, said method comprising:
contacting said gene with a polyamide wherein said polyamide binds at one or more of said repeats, thereby modulating said transcription.

2. The method according to claim 1, wherein said modulating is an increase in transcription.

3. The method according to claim 1, wherein said modulating is a decrease in transcription.

4. The method according to claim 1, wherein said gene is selected from the group consisting of DRPLA, HD, AR, ATXN1, ATXN2, ATXN3, CACNA1A, ATNX7, FMR1, FMR2, FXN, DMPK, SCA8, SCA10, SCA12, NTR, and ZNF9.

5. The method according to claim 4, wherein said gene is FXN.

6. The method according to claim 1, wherein said oligonucleotide sequence consists of 3, 4, or 5 nucleotides.

7. The method according to claim 6, wherein said oligonucleotide sequence is selected from the group consisting of CGG, GCC, GAA, CTG, CAG, CCTG, and ATTCT.

8. The method according to claim 6, wherein said oligonucleotide sequence consists of 3 nucleotides.

9. The method according to claim 8, wherein said oligonucleotide sequence is GAA.

10. The method according to claim 1, wherein the number of said plurality of repeats is in the range 6-1700.

11. The method according to claim 1, wherein the number of said plurality of repeats is in the range 6-34.

12. The method according to claim 1, wherein the number of said plurality of repeats is in the range 35-65.

13. The method according to claim 1, wherein the number of said plurality of repeats is in the range 66-1700.

14. The method according to claim 1, wherein said gene is in a cell.

15. The method according to claim 14, wherein said contacting further comprises localizing said polyamide into the nucleus of said cell.

16. The method according to claim 15, further comprising monitoring said localization.

17. The method according to claim 1, wherein said polyamide comprises a plurality of amide-linked linkable units selected from the group consisting of Im, Py, β, and Dp,
wherein
Im is 1-methyl-1H-imidazole;
Py is 1-methyl-1H-pyrrole;
β is β-alanine; and
Dp is dimethylaminopropylamine.

18. The method according to claim 17, wherein said polyamide is selected from the group consisting of ImImβImImβImβDp, ImPyβImPyβImβDp, and ImβImImβImImβDp.

19. The method according to claim 18, wherein said polyamide is ImPyβImPyβImβDp.

20. A method for treating a subject suffering from a disease associated with a gene of said subject, wherein said gene comprises a plurality of repeats of an oligonucleotide sequence, said method comprising:
administering to said subject a therapeutically effective amount of a polyamide which binds to one or more of said oligonucleotide sequences, thereby modulating the transcription of said gene.

21. The method according to claim 20, wherein said subject is a human.

22. The method according to claim 20, wherein said disease is selected from the group consisting of dentatorubropallidoluysian atrophy, Huntington's disease, spinobulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 8, spinocerebellar ataxia type 10, spinocerebellar ataxia type 12, fragile X syndrome, fragile XE syndrome, Friedreich's ataxia, and myotonic dystrophy type 1, and myotonic dystrophy type 2.

23. The method according to claim 22, wherein said disease is Friedreich's ataxia.
